SEQUENCE LISTING (359 sequences)

SEQ ID NO: 1 - DNA, 24 bp, Artificial sequence
  Primer
tcatgactgcttttgtacaaatca

SEQ ID NO: 2 - DNA, 19 bp, Artificial sequence
  Primer
agggccattgttcaaatat

SEQ ID NO: 3 - DNA, 18 bp, Artificial sequence
  Primer
gagctttctgttgctact

SEQ ID NO: 4 - DNA, 19 bp, Artificial sequence
  Primer
ccctctactccagtctcta

SEQ ID NO: 5 - DNA, 26 bp, Artificial sequence
  Primer
ttcagttaggaccttaaaagagttgt

SEQ ID NO: 6 - DNA, 25 bp, Artificial sequence
  Primer
gctgtgtgtttctttgttctatttg

SEQ ID NO: 7 - DNA, 23 bp, Artificial sequence
  Primer
tgatctgactgaagagtagtacg

SEQ ID NO: 8 - DNA, 25 bp, Artificial sequence
  Primer
ctatctcaccagagtaagagtttca

SEQ ID NO: 9 - DNA, 20 bp, Artificial sequence
  Primer
gtggagaggaggagatgtat

SEQ ID NO: 10 - DNA, 24 bp, Artificial sequence
  Primer
ttctcattgtagtctatctgtgtg

SEQ ID NO: 11 - DNA, 23 bp, Artificial sequence
  Primer
ctccaaggttagaatggctaaag

SEQ ID NO: 12 - DNA, 23 bp, Artificial sequence
  Primer
atatgatttagcctcaaagctgt

SEQ ID NO: 13 - DNA, 20 bp, Artificial sequence
  Primer
tctttgagtcctacgtcctt

SEQ ID NO: 14 - DNA, 25 bp, Artificial sequence
  Primer
ttacgttttgttttcttggaattca

SEQ ID NO: 15 - DNA, 18 bp, Artificial sequence
  Primer
accctcgatttgcatacg

SEQ ID NO: 16 - DNA, 25 bp, Artificial sequence
  Primer
aaagtgggaatacattatagtcagc

SEQ ID NO: 17 - DNA, 19 bp, Artificial sequence
  Primer
tggagaaaggaccaacata

SEQ ID NO: 18 - DNA, 26 bp, Artificial sequence
  Primer
ttgacacactttttagaactaacagt

SEQ ID NO: 19 - DNA, 18 bp, Artificial sequence
  Primer
gcattcacagctgttggt

SEQ ID NO: 20 - DNA, 21 bp, Artificial sequence
  Primer
gaaggaacacaaatgctaact

SEQ ID NO: 21 - DNA, 22 bp, Artificial sequence
  Primer
caagtctcatttgtcaaagtgc

SEQ ID NO: 22 - DNA, 18 bp, Artificial sequence
  Primer
ccagtgcctagaccattt

SEQ ID NO: 23 - DNA, 26 bp, Artificial sequence
  Primer
acatacaattcattcattatctggac

SEQ ID NO: 24 - DNA, 26 bp, Artificial sequence
  Primer
ctgaaaatttggtcatatatcaacct

SEQ ID NO: 25 - DNA, 26 bp, Artificial sequence
  Primer
tggaattaagtttgtggaagctaaga

SEQ ID NO: 26 - DNA, 24 bp, Artificial sequence
  Primer
ctatattcctgtacattgtccagt

SEQ ID NO: 27 - DNA, 22 bp, Artificial sequence
  Primer
tgacaattttgtgtcctgatac

SEQ ID NO: 28 - DNA, 24 bp, Artificial sequence
  Primer
tttatttcctgacacaagcaacca

SEQ ID NO: 29 - DNA, 21 bp, Artificial sequence
  Primer
ctaccactctctgttgacgat

SEQ ID NO: 30 - DNA, 26 bp, Artificial sequence
  Primer
cacaaagaccatttcttttaaaccaa

SEQ ID NO: 31 - DNA, 18 bp, Artificial sequence
  Primer
attggagacaaggctgaa

SEQ ID NO: 32 - DNA, 23 bp, Artificial sequence
  Primer
tagcagttagggaaacattctct

SEQ ID NO: 33 - DNA, 26 bp, Artificial sequence
  Primer
caagcaggattattagaaaatactcc

SEQ ID NO: 34 - DNA, 26 bp, Artificial sequence
  Primer
gtgtaaggaagcagaaacagacttta

SEQ ID NO: 35 - DNA, 25 bp, Artificial sequence
  Primer
ctcagtctctctctctccatataag

SEQ ID NO: 36 - DNA, 24 bp, Artificial sequence
  Primer
tcaagtttcttcaactctgttgct

SEQ ID NO: 37 - DNA, 20 bp, Artificial sequence
  Primer
tatggcagactggagtgttt

SEQ ID NO: 38 - DNA, 25 bp, Artificial sequence
  Primer
ctagttccatgaacatttgagaaat

SEQ ID NO: 39 - DNA, 26 bp, Artificial sequence
  Primer
ggagtgtaaataagtacaaatactgt

SEQ ID NO: 40 - DNA, 26 bp, Artificial sequence
  Primer
catgcttgatcaggataataaacaac

SEQ ID NO: 41 - DNA, 24 bp, Artificial sequence
  Primer
cttggccatcacaaatttcaagaa

SEQ ID NO: 42 - DNA, 19 bp, Artificial sequence
  Primer
agaatgaaacccagcactt

SEQ ID NO: 43 - DNA, 24 bp, Artificial sequence
  Primer
aatagtcaaaaagtgcttacctgt

SEQ ID NO: 44 - DNA, 26 bp, Artificial sequence
  Primer
agacattttgtattttaggcataggt

SEQ ID NO: 45 - DNA, 23 bp, Artificial sequence
  Primer
tgaggtacttttgagtcacccat

SEQ ID NO: 46 - DNA, 24 bp, Artificial sequence
  Primer
agaaatgcaggactgatgatagtt

SEQ ID NO: 47 - DNA, 22 bp, Artificial sequence
  Primer
ttgcatatagtcccatgacagt

SEQ ID NO: 48 - DNA, 18 bp, Artificial sequence
  Primer
caacagtctagaaatgcg

SEQ ID NO: 49 - DNA, 20 bp, Artificial sequence
  Primer
tgccagtggaacacgacaga

SEQ ID NO: 50 - DNA, 24 bp, Artificial sequence
  Primer
tcccataatggagcagtaatcagc

SEQ ID NO: 51 - DNA, 18 bp, Artificial sequence
  Primer
gaccctgaggattgttga

SEQ ID NO: 52 - DNA, 25 bp, Artificial sequence
  Primer
tggcaaatgtattcaacattctcag

SEQ ID NO: 53 - DNA, 19 bp, Artificial sequence
  Primer
gcacccaggtagtatcttc

SEQ ID NO: 54 - DNA, 25 bp, Artificial sequence
  Primer
ataaaacttatcgctatgtgaatgc

SEQ ID NO: 55 - DNA, 22 bp, Artificial sequence
  Primer
agagtccaaagaataatgggca

SEQ ID NO: 56 - DNA, 24 bp, Artificial sequence
  Primer
catgtgatttatttgcctgtctca

SEQ ID NO: 57 - DNA, 23 bp, Artificial sequence
  Primer
actacatgccactttgccataac

SEQ ID NO: 58 - DNA, 25 bp, Artificial sequence
  Primer
aatatcctagatcaggagcaagcaa

SEQ ID NO: 59 - DNA, 25 bp, Artificial sequence
  Primer
aatgtcataattatcagaccaggaa

SEQ ID NO: 60 - DNA, 24 bp, Artificial sequence
  Primer
gagagagagaatggaagagcaata

SEQ ID NO: 61 - DNA, 18 bp, Artificial sequence
  Primer
gacacaccaagagtctca

SEQ ID NO: 62 - DNA, 22 bp, Artificial sequence
  Primer
ccaaaagtgagagtgaaaccct

SEQ ID NO: 63 - DNA, 26 bp, Artificial sequence
  Primer
aggtcatgaaatacaatattgctgct

SEQ ID NO: 64 - DNA, 26 bp, Artificial sequence
  Primer
gagttggttgtttgatgaaaaagtca

SEQ ID NO: 65 - DNA, 26 bp, Artificial sequence
  Primer
aaatgcaataccagaagaagagaaag

SEQ ID NO: 66 - DNA, 26 bp, Artificial sequence
  Primer
acactttacaaaaatgtgcaagacct

SEQ ID NO: 67 - DNA, 20 bp, Artificial sequence
  Primer
agcaacacataaaacaccga

SEQ ID NO: 68 - DNA, 26 bp, Artificial sequence
  Primer
ttccataaatatttgtgactgactga

SEQ ID NO: 69 - DNA, 19 bp, Artificial sequence
  Primer
actgtcacttgtctcccat

SEQ ID NO: 70 - DNA, 20 bp, Artificial sequence
  Primer
gtacagtgaacacatgtcca

SEQ ID NO: 71 - DNA, 24 bp, Artificial sequence
  Primer
gtgatcaacattatgagtagtgct

SEQ ID NO: 72 - DNA, 21 bp, Artificial sequence
  Primer
ctattaaactggatggttttt

SEQ ID NO: 73 - DNA, 21 bp, Artificial sequence
  Primer
caaacagtgttaagctagttt

SEQ ID NO: 74 - DNA, 21 bp, Artificial sequence
  Primer
accaaatttggtagttccact

SEQ ID NO: 75 - DNA, 19 bp, Artificial sequence
  Primer
actccggcaggtttctgtt

SEQ ID NO: 76 - DNA, 21 bp, Artificial sequence
  Primer
atgttcctctctattgattct

SEQ ID NO: 77 - DNA, 26 bp, Artificial sequence
  Primer
ttcagcaagatagatacagaaggaac

SEQ ID NO: 78 - DNA, 24 bp, Artificial sequence
  Primer
gttttaacttttgcacagattctg

SEQ ID NO: 79 - DNA, 25 bp, Artificial sequence
  Primer
gattgaaatgctagagtgaatttgt

SEQ ID NO: 80 - DNA, 26 bp, Artificial sequence
  Primer
gtctatcttttcatgagtttgttgga

SEQ ID NO: 81 - DNA, 18 bp, Artificial sequence
  Primer
agtgctgagctacctctt

SEQ ID NO: 82 - DNA, 18 bp, Artificial sequence
  Primer
actttggagcaaaggtca

SEQ ID NO: 83 - DNA, 22 bp, Artificial sequence
  Primer
tcatctttgcatatcccttcca

SEQ ID NO: 84 - DNA, 23 bp, Artificial sequence
  Primer
tcaggttttcttaactcttctga

SEQ ID NO: 85 - DNA, 20 bp, Artificial sequence
  Primer
ttaccatggcctaggtccta

SEQ ID NO: 86 - DNA, 23 bp, Artificial sequence
  Primer
tcatttgagaactttctttttca

SEQ ID NO: 87 - DNA, 24 bp, Artificial sequence
  Primer
catttccagaaacattccatttct

SEQ ID NO: 88 - DNA, 22 bp, Artificial sequence
  Primer
atacccttcagatgcagagcat

SEQ ID NO: 89 - DNA, 21 bp, Artificial sequence
  Primer
caaaattctgaatcggccaaa

SEQ ID NO: 90 - DNA, 25 bp, Artificial sequence
  Primer
atctttaacattgccaattaggtca

SEQ ID NO: 91 - DNA, 25 bp, Artificial sequence
  Primer
atgtatatttgacccatacatgagt

SEQ ID NO: 92 - DNA, 22 bp, Artificial sequence
  Primer
ttagaaactcaggaagacagga

SEQ ID NO: 93 - DNA, 26 bp, Artificial sequence
  Primer
ttgtgaagttaaattctccactctgt

SEQ ID NO: 94 - DNA, 24 bp, Artificial sequence
  Primer
tatgtcaactggattaaggaaaaa

SEQ ID NO: 95 - DNA, 26 bp, Artificial sequence
  Primer
taatacatgttccatttgccaatgag

SEQ ID NO: 96 - DNA, 18 bp, Artificial sequence
  Primer
cccattctcttcacttgt

SEQ ID NO: 97 - DNA, 19 bp, Artificial sequence
  Primer
tctggctatgtaagtggct

SEQ ID NO: 98 - DNA, 25 bp, Artificial sequence
  Primer
aatcagtttttctctttcttactcc

SEQ ID NO: 99 - DNA, 23 bp, Artificial sequence
  Primer
cacacgcatacacacatataatg

SEQ ID NO: 100 - DNA, 25 bp, Artificial sequence
  Primer
agactttgaggaagaattcaacagt

SEQ ID NO: 101 - DNA, 24 bp, Artificial sequence
  Primer
taaattgctttgtgagtgcctact

SEQ ID NO: 102 - DNA, 26 bp, Artificial sequence
  Primer
gttacttcaaatttgaatgaccaaag

SEQ ID NO: 103 - DNA, 25 bp, Artificial sequence
  Primer
aaaatagtgctgataacaaggtggt

SEQ ID NO: 104 - DNA, 26 bp, Artificial sequence
  Primer
tccatcttatttgatcctaactggaa

SEQ ID NO: 105 - DNA, 18 bp, Artificial sequence
  Primer
catactgcagcactgaca

SEQ ID NO: 106 - DNA, 18 bp, Artificial sequence
  Primer
ttgcactccatggcattg

SEQ ID NO: 107 - DNA, 20 bp, Artificial sequence
  Primer
aatgtggagacctcgagatt

SEQ ID NO: 108 - DNA, 18 bp, Artificial sequence
  Primer
tgtgaatgggttagcata

SEQ ID NO: 109 - DNA, 14 bp, Artificial sequence
  Primer
ggcaggcctcgaat

SEQ ID NO: 110 - DNA, 17 bp, Artificial sequence
  Primer
cgctcctgacacatttc

SEQ ID NO: 111 - DNA, 18 bp, Artificial sequence
  Primer
cctccaatttccctcaac

SEQ ID NO: 112 - DNA, 22 bp, Artificial sequence
  Primer
aactcagtctcttctttcttgg

SEQ ID NO: 113 - DNA, 24 bp, Artificial sequence
  Primer
atgcttccagtttatttcccttct

SEQ ID NO: 114 - DNA, 18 bp, Artificial sequence
  Primer
ctggtctctggaatacaa

SEQ ID NO: 115 - DNA, 18 bp, Artificial sequence
  Primer
ttctggtgtcagcacact

SEQ ID NO: 116 - DNA, 19 bp, Artificial sequence
  Primer
gattagaacccgagtcgta

SEQ ID NO: 117 - DNA, 22 bp, Artificial sequence
  Primer
aagactgaacataatgactgac

SEQ ID NO: 118 - DNA, 18 bp, Artificial sequence
  Primer
gcaggtccttaaggacag

SEQ ID NO: 119 - DNA, 18 bp, Artificial sequence
  Primer
cccacctcctttcacaca

SEQ ID NO: 120 - DNA, 18 bp, Artificial sequence
  Primer
taggccatgaggagaaag

SEQ ID NO: 121 - DNA, 18 bp, Artificial sequence
  Primer
ctactcacttccttggaa

SEQ ID NO: 122 - DNA, 18 bp, Artificial sequence
  Primer
ccaagccttgtagcactt

SEQ ID NO: 123 - DNA, 18 bp, Artificial sequence
  Primer
accaacagctgggtgaaa

SEQ ID NO: 124 - DNA, 18 bp, Artificial sequence
  Primer
caacccagattcagctag

SEQ ID NO: 125 - DNA, 25 bp, Artificial sequence
  Primer
agaacctttcttacccttcctaaga

SEQ ID NO: 126 - DNA, 22 bp, Artificial sequence
  Primer
tctgctttacaatgacttgcct

SEQ ID NO: 127 - DNA, 21 bp, Artificial sequence
  Primer
agtactccagtagaaaccaga

SEQ ID NO: 128 - DNA, 25 bp, Artificial sequence
  Primer
agactctagccacagttagttttgg

SEQ ID NO: 129 - DNA, 21 bp, Artificial sequence
  Primer
agcccttgtttcttcctctct

SEQ ID NO: 130 - DNA, 18 bp, Artificial sequence
  Primer
atgtgcctcagacactga

SEQ ID NO: 131 - DNA, 18 bp, Artificial sequence
  Primer
tctaggtgccagtgtttg

SEQ ID NO: 132 - DNA, 23 bp, Artificial sequence
  Primer
aagttgctaaaaaggcaaagaat

SEQ ID NO: 133 - DNA, 19 bp, Artificial sequence
  Primer
caagacctagaagcacctt

SEQ ID NO: 134 - DNA, 18 bp, Artificial sequence
  Primer
aggccaatcactggtgaa

SEQ ID NO: 135 - DNA, 18 bp, Artificial sequence
  Primer
ctctccttgttctcagca

SEQ ID NO: 136 - DNA, 18 bp, Artificial sequence
  Primer
ggaaggcatgttagtgaa

SEQ ID NO: 137 - DNA, 17 bp, Artificial sequence
  Primer
gccgcacatacgtgaca

SEQ ID NO: 138 - DNA, 18 bp, Artificial sequence
  Primer
aggtggttttcctgacat

SEQ ID NO: 139 - DNA, 20 bp, Artificial sequence
  Primer
tctttaatggctgtgcgtta

SEQ ID NO: 140 - DNA, 18 bp, Artificial sequence
  Primer
aatggtcgggattgacac

SEQ ID NO: 141 - DNA, 18 bp, Artificial sequence
  Primer
tcatgcacagaaagcaat

SEQ ID NO: 142 - DNA, 18 bp, Artificial sequence
  Primer
tttgggtgggtgattttt

SEQ ID NO: 143 - DNA, 27 bp, Artificial sequence
  Primer
tgagcatatttaatatcagccacaaca

SEQ ID NO: 144 - DNA, 19 bp, Artificial sequence
  Primer
ggcagagagtaaccaggtt

SEQ ID NO: 145 - DNA, 19 bp, Artificial sequence
  Primer
aaacggaacgagaaaatgc

SEQ ID NO: 146 - DNA, 18 bp, Artificial sequence
  Primer
acgatcagggagcagaaa

SEQ ID NO: 147 - DNA, 18 bp, Artificial sequence
  Primer
gctctgctgttttagagg

SEQ ID NO: 148 - DNA, 24 bp, Artificial sequence
  Primer
aagaagccaatactgaaccaaact

SEQ ID NO: 149 - DNA, 18 bp, Artificial sequence
  Primer
cagtcagtcctgcatctt

SEQ ID NO: 150 - DNA, 16 bp, Artificial sequence
  Primer
gcagcctcttgatctc

SEQ ID NO: 151 - DNA, 21 bp, Artificial sequence
  Primer
agcatccaagagcctcagagt

SEQ ID NO: 152 - DNA, 18 bp, Artificial sequence
  Primer
actgtggagttgacacag

SEQ ID NO: 153 - DNA, 18 bp, Artificial sequence
  Primer
atggcatagaatgtggct

SEQ ID NO: 154 - DNA, 19 bp, Artificial sequence
  Primer
gcagtatctcacactgaca

SEQ ID NO: 155 - DNA, 16 bp, Artificial sequence
  Primer
gaggcggatctgcttg

SEQ ID NO: 156 - DNA, 18 bp, Artificial sequence
  Primer
ctctgtgtccataccacc

SEQ ID NO: 157 - DNA, 26 bp, Artificial sequence
  Primer
actttttacccaaaacctagtctcta

SEQ ID NO: 158 - DNA, 16 bp, Artificial sequence
  Primer
gccatccagtccctac

SEQ ID NO: 159 - DNA, 18 bp, Artificial sequence
  Primer
gtacatgagacaggaagc

SEQ ID NO: 160 - DNA, 19 bp, Artificial sequence
  Primer
caggtctcttccactttaa

SEQ ID NO: 161 - DNA, 19 bp, Artificial sequence
  Primer
ttccaggaagcaagctcta

SEQ ID NO: 162 - DNA, 17 bp, Artificial sequence
  Primer
gttgaagtcggcttcac

SEQ ID NO: 163 - DNA, 27 bp, Artificial sequence
  Primer
ctcttgaatactatttttgtttctttg

SEQ ID NO: 164 - DNA, 17 bp, Artificial sequence
  Primer
gaagaccaggtccagta

SEQ ID NO: 165 - DNA, 28 bp, Artificial sequence
  Primer
agtcacttaaaagctgaatgattcagaa

SEQ ID NO: 166 - DNA, 17 bp, Artificial sequence
  Primer
agccaagctgagcattg

SEQ ID NO: 167 - DNA, 15 bp, Artificial sequence
  Primer
gtcgatcttgctgaa

SEQ ID NO: 168 - DNA, 16 bp, Artificial sequence
  Primer
ggtgcaaatgaaccgt

SEQ ID NO: 169 - DNA, 18 bp, Artificial sequence
  Primer
atcaggtctgctacagct

SEQ ID NO: 170 - DNA, 18 bp, Artificial sequence
  Primer
tcagggcaagctgataca

SEQ ID NO: 171 - DNA, 24 bp, Artificial sequence
  Primer
aacaccatctgctaaactaattcc

SEQ ID NO: 172 - DNA, 18 bp, Artificial sequence
  Primer
caccagaggcagaatcag

SEQ ID NO: 173 - DNA, 22 bp, Artificial sequence
  Primer
gtatgccataaagcctttatgt

SEQ ID NO: 174 - DNA, 18 bp, Artificial sequence
  Primer
ctatgctcccttagtcct

SEQ ID NO: 175 - DNA, 23 bp, Artificial sequence
  Primer
catttcttacaaggagccaaaaa

SEQ ID NO: 176 - DNA, 19 bp, Artificial sequence
  Primer
caccagaagatactacctg

SEQ ID NO: 177 - DNA, 26 bp, Artificial sequence
  Primer
aagcaaatactactttttgtcagtaa

SEQ ID NO: 178 - DNA, 25 bp, Artificial sequence
  Primer
aaaaacaaagtggtagtaggaaagg

SEQ ID NO: 179 - DNA, 20 bp, Artificial sequence
  Primer
caaacagacctggaaaagtt

SEQ ID NO: 180 - DNA, 18 bp, Artificial sequence
  Primer
tcttgaaacgccatcaac

SEQ ID NO: 181 - DNA, 25 bp, Artificial sequence
  Primer
gtaaaacgttgagtacagttcttgg

SEQ ID NO: 182 - DNA, 25 bp, Artificial sequence
  Primer
cagtacttcaagattttaggtcatt

SEQ ID NO: 183 - DNA, 23 bp, Artificial sequence
  Primer
ccaaaaacatgaaacatttgacc

SEQ ID NO: 184 - DNA, 26 bp, Artificial sequence
  Primer
taatgaatagccaagaaagttcatgg

SEQ ID NO: 185 - DNA, 24 bp, Artificial sequence
  Primer
tgattcatgacagaatgcttatgg

SEQ ID NO: 186 - DNA, 19 bp, Artificial sequence
  Primer
ctgacatcaaagccaagtt

SEQ ID NO: 187 - DNA, 20 bp, Artificial sequence
  Primer
cttcagcttgttcgatacca

SEQ ID NO: 188 - DNA, 20 bp, Artificial sequence
  Primer
ctaggaaaatgaggatgtga

SEQ ID NO: 189 - DNA, 26 bp, Artificial sequence
  Primer
ggaacaatttcatgaaaagaccaaat

SEQ ID NO: 190 - DNA, 26 bp, Artificial sequence
  Primer
atgacagtagccctagaatatcagtg

SEQ ID NO: 191 - DNA, 26 bp, Artificial sequence
  Primer
ggaataagataatgggcataacatat

SEQ ID NO: 192 - DNA, 24 bp, Artificial sequence
  Primer
gacatgtttctttgagtgtacagt

SEQ ID NO: 193 - DNA, 24 bp, Artificial sequence
  Primer
gatgagaaatccactctggttcat

SEQ ID NO: 194 - DNA, 25 bp, Artificial sequence
  Primer
ggaatcctcatagatgtcagttttt

SEQ ID NO: 195 - DNA, 20 bp, Artificial sequence
  Primer
gccatcgctttcatcataga

SEQ ID NO: 196 - DNA, 21 bp, Artificial sequence
  Primer
agtgctgtggtatggttaaga

SEQ ID NO: 197 - DNA, 22 bp, Artificial sequence
  Primer
ctgtagcaatgtagattcttcc

SEQ ID NO: 198 - DNA, 26 bp, Artificial sequence
  Primer
tgttttccatttcagattctctactt

SEQ ID NO: 199 - DNA, 18 bp, Artificial sequence
  Primer
ctgttgagaaacgcacaa

SEQ ID NO: 200 - DNA, 26 bp, Artificial sequence
  Primer
ccaaatttggtaggtctactctgata

SEQ ID NO: 201 - DNA, 26 bp, Artificial sequence
  Primer
tccaattaagattaaatgagaaactg

SEQ ID NO: 202 - DNA, 26 bp, Artificial sequence
  Primer
aaggaccttaagatcctagaagatta

SEQ ID NO: 203 - DNA, 23 bp, Artificial sequence
  Primer
ctccttgccttgattgaatcata

SEQ ID NO: 204 - DNA, 22 bp, Artificial sequence
  Primer
ttcaaatttgcctccttgctaa

SEQ ID NO: 205 - DNA, 21 bp, Artificial sequence
  Primer
tcagaagaaaattggactggt

SEQ ID NO: 206 - DNA, 26 bp, Artificial sequence
  Primer
tcaggttaagcctcatacgtttaaaa

SEQ ID NO: 207 - DNA, 26 bp, Artificial sequence
  Primer
ttttggcattcttttcccattgacta

SEQ ID NO: 208 - DNA, 21 bp, Artificial sequence
  Primer
gagaagactgacccttggttt

SEQ ID NO: 209 - DNA, 26 bp, Artificial sequence
  Primer
gcaattgttgaaagctttgaaataaa

SEQ ID NO: 210 - DNA, 22 bp, Artificial sequence
  Primer
aaaggcaaaagaatggctactt

SEQ ID NO: 211 - DNA, 25 bp, Artificial sequence
  Primer
atggaaaacccaggttagttattat

SEQ ID NO: 212 - DNA, 18 bp, Artificial sequence
  Primer
gcagacactgccttgaag

SEQ ID NO: 213 - DNA, 25 bp, Artificial sequence
  Primer
atctgcaaaatggagagaatacaat

SEQ ID NO: 214 - DNA, 24 bp, Artificial sequence
  Primer
tggcttgttgtactctaattgagc

SEQ ID NO: 215 - DNA, 26 bp, Artificial sequence
  Primer
tagaacagcctaatatagcaagacac

SEQ ID NO: 216 - DNA, 26 bp, Artificial sequence
  Primer
aacgttttagaatctgtgttatgagt

SEQ ID NO: 217 - DNA, 21 bp, Artificial sequence
  Primer
aaagaggaaaccaggtgagtt

SEQ ID NO: 218 - DNA, 18 bp, Artificial sequence
  Primer
tgtcctgtcagacaacca

SEQ ID NO: 219 - DNA, 22 bp, Artificial sequence
  Primer
ttgctacttcagttcttcctgt

SEQ ID NO: 220 - DNA, 17 bp, Artificial sequence
  Primer
ggtgcaaagagcgggat

SEQ ID NO: 221 - DNA, 25 bp, Artificial sequence
  Primer
aagctttatggcttgatataatccc

SEQ ID NO: 222 - DNA, 23 bp, Artificial sequence
  Primer
atttttacctgccacaggtatag

SEQ ID NO: 223 - DNA, 21 bp, Artificial sequence
  Primer
ggagagaaaatcagtttgagc

SEQ ID NO: 224 - DNA, 22 bp, Artificial sequence
  Primer
aatcaatcattgtccagttgga

SEQ ID NO: 225 - DNA, 20 bp, Artificial sequence
  Primer
actgctctcagaagtgaatg

SEQ ID NO: 226 - DNA, 24 bp, Artificial sequence
  Primer
acattatacacacatttctcactc

SEQ ID NO: 227 - DNA, 26 bp, Artificial sequence
  Primer
tacatactggtgcttttacttctgtt

SEQ ID NO: 228 - DNA, 24 bp, Artificial sequence
  Primer
atgctgattaacaggataagctga

SEQ ID NO: 229 - DNA, 23 bp, Artificial sequence
  Primer
cttattcattccacatcctggta

SEQ ID NO: 230 - DNA, 24 bp, Artificial sequence
  Primer
taatgtcacccactaaactcgaca

SEQ ID NO: 231 - DNA, 22 bp, Artificial sequence
  Primer
gttccacagtttttggacattc

SEQ ID NO: 232 - DNA, 21 bp, Artificial sequence
  Primer
gataggaagaatgactgggtt

SEQ ID NO: 233 - DNA, 22 bp, Artificial sequence
  Primer
ctactacccattctagatccct

SEQ ID NO: 234 - DNA, 26 bp, Artificial sequence
  Primer
tataggaatcctcaggtgatgtttca

SEQ ID NO: 235 - DNA, 26 bp, Artificial sequence
  Primer
taccatgtgacaatgaagattcacaa

SEQ ID NO: 236 - DNA, 25 bp, Artificial sequence
  Primer
gtctacttaatggatgtcgaattcc

SEQ ID NO: 237 - DNA, 21 bp, Artificial sequence
  Primer
tctgggtttgtggttgttgtt

SEQ ID NO: 238 - DNA, 26 bp, Artificial sequence
  Primer
ccaactttgctctctttgatttttat

SEQ ID NO: 239 - DNA, 22 bp, Artificial sequence
  Primer
tgcattttctgtctttttgctg

SEQ ID NO: 240 - DNA, 23 bp, Artificial sequence
  Primer
tgagatagagagaagagaggttt

SEQ ID NO: 241 - DNA, 19 bp, Artificial sequence
  Primer
tctattgtgctgaccactt

SEQ ID NO: 242 - DNA, 25 bp, Artificial sequence
  Primer
ccatggtaatatattccacatccaa

SEQ ID NO: 243 - DNA, 23 bp, Artificial sequence
  Primer
gtgctctttcttctacctactct

SEQ ID NO: 244 - DNA, 26 bp, Artificial sequence
  Primer
tttattttggttcttcactgtccctt

SEQ ID NO: 245 - DNA, 22 bp, Artificial sequence
  Primer
aagccctgtaacttttctgctc

SEQ ID NO: 246 - DNA, 19 bp, Artificial sequence
  Primer
tttctcagccctcagtgtt

SEQ ID NO: 247 - DNA, 23 bp, Artificial sequence
  Primer
caaaatgcaaagcccagatattt

SEQ ID NO: 248 - DNA, 22 bp, Artificial sequence
  Primer
ctctcttctgatcctttcaggt

SEQ ID NO: 249 - DNA, 26 bp, Artificial sequence
  Primer
caaaccatatcagtatctaaaaggaa

SEQ ID NO: 250 - DNA, 24 bp, Artificial sequence
  Primer
cagatagcaataacagaattggcc

SEQ ID NO: 251 - DNA, 25 bp, Artificial sequence
  Primer
actataatctactagccaacaccat

SEQ ID NO: 252 - DNA, 26 bp, Artificial sequence
  Primer
aaattgttatgatactggatttggca

SEQ ID NO: 253 - DNA, 26 bp, Artificial sequence
  Primer
agggaccttaatgtttctcaaaaatt

SEQ ID NO: 254 - DNA, 20 bp, Artificial sequence
  Primer
aatgtcacccaatagctcca

SEQ ID NO: 255 - DNA, 24 bp, Artificial sequence
  Primer
caatggttatgtaaacaggtctct

SEQ ID NO: 256 - DNA, 23 bp, Artificial sequence
  Primer
ttagaattgcccatcaggagctt

SEQ ID NO: 257 - DNA, 23 bp, Artificial sequence
  Primer
ataaatgccaggtggttatgagt

SEQ ID NO: 258 - DNA, 24 bp, Artificial sequence
  Primer
gaacaaactcttccaatttacctg

SEQ ID NO: 259 - DNA, 18 bp, Artificial sequence
  Primer
tctgcctttagcccaatt

SEQ ID NO: 260 - DNA, 20 bp, Artificial sequence
  Primer
ctggcataaccctgtagtat

SEQ ID NO: 261 - DNA, 26 bp, Artificial sequence
  Primer
catactgcttccaaaattcagtctat

SEQ ID NO: 262 - DNA, 21 bp, Artificial sequence
  Primer
agctgatctccctttgtggaa

SEQ ID NO: 263 - DNA, 25 bp, Artificial sequence
  Primer
aaggaagcagattcaagtaggaatt

SEQ ID NO: 264 - DNA, 26 bp, Artificial sequence
  Primer
caccaatattgcattttccagtttca

SEQ ID NO: 265 - DNA, 19 bp, Artificial sequence
  Primer
taacgaccatggagggtaa

SEQ ID NO: 266 - DNA, 22 bp, Artificial sequence
  Primer
gttgccatagtggagaaccata

SEQ ID NO: 267 - DNA, 24 bp, Artificial sequence
  Primer
tgtctagtaaaatagcctcagtct

SEQ ID NO: 268 - DNA, 23 bp, Artificial sequence
  Primer
ggctgttagactcttcaatattg

SEQ ID NO: 269 - DNA, 26 bp, Artificial sequence
  Primer
tgaattaaccttggaaatccatcttt

SEQ ID NO: 270 - DNA, 25 bp, Artificial sequence
  Primer
ataaccatacaagctcttagaatgg

SEQ ID NO: 271 - DNA, 18 bp, Artificial sequence
  Primer
tgcttggctctgaacact

SEQ ID NO: 272 - DNA, 23 bp, Artificial sequence
  Primer
atacagggtgactgattcacatc

SEQ ID NO: 273 - DNA, 19 bp, Artificial sequence
  Primer
ctacagacctttggtcatt

SEQ ID NO: 274 - DNA, 25 bp, Artificial sequence
  Primer
aatgattcttatatggcaactgcaa

SEQ ID NO: 275 - DNA, 26 bp, Artificial sequence
  Primer
aaatcacacaaattaattgctttgtg

SEQ ID NO: 276 - DNA, 18 bp, Artificial sequence
  Primer
tttgctgaatgccacaag

SEQ ID NO: 277 - DNA, 20 bp, Artificial sequence
  Primer
caaggctttcatttcaaaag

SEQ ID NO: 278 - DNA, 18 bp, Artificial sequence
  Primer
cttcccaccattgtgaag

SEQ ID NO: 279 - DNA, 19 bp, Artificial sequence
  Primer
acaactatgccgctgcttt

SEQ ID NO: 280 - DNA, 18 bp, Artificial sequence
  Primer
ctgctagcaccagctctt

SEQ ID NO: 281 - DNA, 16 bp, Artificial sequence
  Primer
cactgggctatttcca

SEQ ID NO: 282 - DNA, 23 bp, Artificial sequence
  Primer
atggaagatgttcatctaaggga

SEQ ID NO: 283 - DNA, 20 bp, Artificial sequence
  Primer
ggagtataggcagtgtgtgt

SEQ ID NO: 284 - DNA, 25 bp, Artificial sequence
  Primer
aacaccacagaacaagttctttgag

SEQ ID NO: 285 - DNA, 20 bp, Artificial sequence
  Primer
tggctggctttatttggtta

SEQ ID NO: 286 - DNA, 20 bp, Artificial sequence
  Primer
cacgaggatcaatcttttct

SEQ ID NO: 287 - DNA, 18 bp, Artificial sequence
  Primer
gaatgggtcttggcaatg

SEQ ID NO: 288 - DNA, 19 bp, Artificial sequence
  Primer
tacatggtcaccggaaatc

SEQ ID NO: 289 - DNA, 19 bp, Artificial sequence
  Primer
gtcaagctgctcacattta

SEQ ID NO: 290 - DNA, 24 bp, Artificial sequence
  Primer
atggagttgtaggttatgagaagg

SEQ ID NO: 291 - DNA, 18 bp, Artificial sequence
  Primer
ggtgtcaacaggaacatg

SEQ ID NO: 292 - DNA, 21 bp, Artificial sequence
  Primer
agtcattgctcttcagtgcta

SEQ ID NO: 293 - DNA, 20 bp, Artificial sequence
  Primer
gcttgtaaagactttttggg

SEQ ID NO: 294 - DNA, 19 bp, Artificial sequence
  Primer
gtgctgtgatgagtatgag

SEQ ID NO: 295 - DNA, 18 bp, Artificial sequence
  Primer
aaatgcccagaccagtga

SEQ ID NO: 296 - DNA, 26 bp, Artificial sequence
  Primer
tctctgtctccatcatcatcacttac

SEQ ID NO: 297 - DNA, 24 bp, Artificial sequence
  Primer
ttatttattgccacattctcagca

SEQ ID NO: 298 - DNA, 18 bp, Artificial sequence
  Primer
gtcacttggagaacgtac

SEQ ID NO: 299 - DNA, 18 bp, Artificial sequence
  Primer
cacccagagtattctgtg

SEQ ID NO: 300 - DNA, 20 bp, Artificial sequence
  Primer
ccaccagcagacctagaatt

SEQ ID NO: 301 - DNA, 25 bp, Artificial sequence
  Primer
agtctcttgaatttagtcacagact

SEQ ID NO: 302 - DNA, 21 bp, Artificial sequence
  Primer
tttcatggttctgcagattgt

SEQ ID NO: 303 - DNA, 19 bp, Artificial sequence
  Primer
ttcagcctgcttttgtttg

SEQ ID NO: 304 - DNA, 20 bp, Artificial sequence
  Primer
ccaccttcctgagagaagag

SEQ ID NO: 305 - DNA, 22 bp, Artificial sequence
  Primer
taatattggtgacgcccatagt

SEQ ID NO: 306 - DNA, 24 bp, Artificial sequence
  Primer
gatgattaaccatgttgaatcagc

SEQ ID NO: 307 - DNA, 18 bp, Artificial sequence
  Primer
gagaggtttgagctgatg

SEQ ID NO: 308 - DNA, 18 bp, Artificial sequence
  Primer
tagaaggtgggagagaca

SEQ ID NO: 309 - DNA, 17 bp, Artificial sequence
  Primer
agggagacactaacgga

SEQ ID NO: 310 - DNA, 18 bp, Artificial sequence
  Primer
tctgcatgtagtaggcat

SEQ ID NO: 311 - DNA, 18 bp, Artificial sequence
  Primer
gatcctgtgacacgtact

SEQ ID NO: 312 - DNA, 18 bp, Artificial sequence
  Primer
gtggaggcagcgagtata

SEQ ID NO: 313 - DNA, 18 bp, Artificial sequence
  Primer
gacgtccatgcagttttg

SEQ ID NO: 314 - DNA, 19 bp, Artificial sequence
  Primer
gacagggtatgagagtgag

SEQ ID NO: 315 - DNA, 22 bp, Artificial sequence
  Primer
ggagtttctgaatcattcagct

SEQ ID NO: 316 - DNA, 18 bp, Artificial sequence
  Primer
ggctcaagtctcagacaa

SEQ ID NO: 317 - DNA, 19 bp, Artificial sequence
  Primer
tgcccttgtactcacgaag

SEQ ID NO: 318 - DNA, 18 bp, Artificial sequence
  Primer
agagtggagggaggatct

SEQ ID NO: 319 - DNA, 18 bp, Artificial sequence
  Primer
cttgctgtccaacattcc

SEQ ID NO: 320 - DNA, 20 bp, Artificial sequence
  Primer
gaactgacaaaagctggttg

SEQ ID NO: 321 - DNA, 26 bp, Artificial sequence
  Primer
catttcctttcattgtttccttttgg

SEQ ID NO: 322 - DNA, 15 bp, Artificial sequence
  Primer
tcgtcctggaaggat

SEQ ID NO: 323 - DNA, 19 bp, Artificial sequence
  Primer
atgaatgtgcaggaactct

SEQ ID NO: 324 - DNA, 24 bp, Artificial sequence
  Primer
cctatagcatagctgaatacttac

SEQ ID NO: 325 - DNA, 18 bp, Artificial sequence
  Primer
cagggccactcagtttat

SEQ ID NO: 326 - DNA, 18 bp, Artificial sequence
  Primer
aatggctctgttgtgtac

SEQ ID NO: 327 - DNA, 19 bp, Artificial sequence
  Primer
gaggtcttgaaatacacac

SEQ ID NO: 328 - DNA, 19 bp, Artificial sequence
  Primer
gcttgcttcctggaatgtc

SEQ ID NO: 329 - DNA, 24 bp, Artificial sequence
  Primer
cttttagttaaaaatgaggcttcc

SEQ ID NO: 330 - DNA, 18 bp, Artificial sequence
  Primer
gactaagagccagagttc

SEQ ID NO: 331 - DNA, 20 bp, Artificial sequence
  Primer
ctatgtctccactgttaacc

SEQ ID NO: 332 - DNA, 20 bp, Artificial sequence
  Primer
gcctgtgttcaattctaggg

SEQ ID NO: 333 - DNA, 18 bp, Artificial sequence
  Primer
cctcaccagcagcaacct

SEQ ID NO: 334 - DNA, 22 bp, Artificial sequence
  Primer
agaagaaggtcattgtgatccc

SEQ ID NO: 335 - DNA, 24 bp, Artificial sequence
  Primer
gctgtattcagatgctggatataa

SEQ ID NO: 336 - DNA, 19 bp, Artificial sequence
  Primer
aggttcttcctgaaccatt

SEQ ID NO: 337 - DNA, 15 bp, Artificial sequence
  Primer
ttccgtttagggcct

SEQ ID NO: 338 - DNA, 15 bp, Artificial sequence
  Primer
ttcgtgccctaacag

SEQ ID NO: 339 - DNA, 17 bp, Artificial sequence
  Primer
tgcacgatttctactgc

SEQ ID NO: 340 - DNA, 16 bp, Artificial sequence
  Primer
aggttctccgaggagg

SEQ ID NO: 341 - DNA, 18 bp, Artificial sequence
  Primer
tcctggttatcccacaca

SEQ ID NO: 342 - DNA, 26 bp, Artificial sequence
  Primer
ggaaatgtccttgagaattagaatta

SEQ ID NO: 343 - DNA, 19 bp, Artificial sequence
  Primer
atggccttttctactgtct

SEQ ID NO: 344 - DNA, 19 bp, Artificial sequence
  Primer
gatggccttttcttttctt

SEQ ID NO: 345 - DNA, 17 bp, Artificial sequence
  Primer
acggtttagtcacaagt

SEQ ID NO: 346 - DNA, 15 bp, Artificial sequence
  Primer
gtcacttaggctcag

SEQ ID NO: 347 - DNA, 16 bp, Artificial sequence
  Primer
tcaactccatctccat

SEQ ID NO: 348 - DNA, 16 bp, Artificial sequence
  Primer
gtcttttggagaagtc

SEQ ID NO: 349 - DNA, 18 bp, Artificial sequence
  Primer
cattgtgttcttgtagtc

SEQ ID NO: 350 - DNA, 16 bp, Artificial sequence
  Primer
attgcttatttatatc

SEQ ID NO: 351 - DNA, 15 bp, Artificial sequence
  Primer
caactggtactcatc

SEQ ID NO: 352 - DNA, 15 bp, Artificial sequence
  Primer
ttacaatccaacact

SEQ ID NO: 353 - DNA, 20 bp, Artificial sequence
  Primer
cccccagtcacttaggctca

SEQ ID NO: 354 - DNA, 19 bp, Artificial sequence
  Primer
ctttcaactccatctccat

SEQ ID NO: 355 - DNA, 29 bp, Artificial sequence
  Primer
actgaacttgtttatcaaatctacgtgtc

SEQ ID NO: 356 - DNA, 28 bp, Artificial sequence
  Primer
cattgtgttcttgtagtcagagtgtact

SEQ ID NO: 357 - DNA, 26 bp, Artificial sequence
  Primer
tcttgagtctgcaactggtactcatc

SEQ ID NO: 358 - DNA, 24 bp, Artificial sequence
  Primer
tgcttctctttctgtgtacccttc

SEQ ID NO: 359 - DNA, 23 bp, Artificial sequence
  Primer
gattgcttatttatatctccaag

(19) United States
(12) Patent Application Publication
(10) Pub. No.: US 2022/0333202 A1
(43) Pub. Date: Oct. 20, 2022

(54) METHOD OF DIAGNOSIS OF HEMOPHILIA

(71) Applicant: Machaon Diagnostics, Inc., Berkeley, CA (US)

(72) Inventors: James S. Kain, Berkeley, CA (US); Michael P. Ero, Berkeley, CA (US)

(73) Assignee: Machaon Diagnostics, Inc., Berkeley, CA (US)

(21) Appl. No.: 17/840,801

(22) Filed: Jun. 15, 2022

Related U.S. Application Data
(63) Continuation-in-part of application No. 16/676,041, filed on Nov. 6, 2019.

(51) Int. Cl. C12Q 1/6883; C12Q 1/6874

(52) U.S. Cl. CPC C12Q 1/6883; C12Q 1/6874; C12Q 2600/118; C12Q 2600/156

(57) ABSTRACT

A method for determining a subject's risk for developing hemophilia A, hemophilia B, or von Willebrand disease (VWD) is described. The method involves obtaining a sample of genetic material from the subject. The genetic material is amplifed using primers specific for the genes underlying hemophilia A, hemophilia B and VWD. The DNA sequence of the amplified genetic material is determined and compared with a DNA sequence from a normal control subject. One or more DNA sequence alterations in the amplified genetic material not present in the DNA sequence from the normal control subject indicates that the subject is at risk for developing hemophilia A, hemophilia B, or VWD.

### CROSS REFERENCE TO RELATED APPLICATIONS

This application is a continuation-in-part of U.S. patent application Ser. No. 16/676,041, filed on Nov. 6, 2019, the contents of which are incorporated herein by reference in its entirety.

### SEQUENCE LISTING

The present application is being filed along with a Sequence Listing in ASCII text format in lieu of a paper copy. The Sequence Listing is provided as a file titled “Sequence listing.txt,” created Jun. 14, 2022, and is 64 kilobytes in size. The information in the electronic format of the Sequence Listing is incorporated herein by reference in its entirety.

### BACKGROUND

Bleeding disorders are a group of conditions that feature spontaneous internal bleeding due to lack of functional clotting factors. Perhaps the most famous bleeding disorder is hemophilia, of which there are several types. Hemophilia A and hemophilia B are both chromosome X-linked recessive bleeding disorders caused by genetic defects found in the human coagulation factor VIII (F8) and IX (F9) genes, respectively. While hemophilia may be the most well-known bleeding disorder, von Willebrand disease (VWD), caused by genetic defects in the von Willebrand factor (VWF) gene, is thought to be the most common inherited bleeding disorder. Despite its frequency, only a handful of patients are diagnosed since the symptoms are often mild. VWD can exhibit either an autosomal recessive (type 2N and 3) or an autosomal dominant (type 1, 2A, 2B and 2M) inheritance pattern. Type 2N patients can present clinically as hemophilia A and are often misdiagnosed as hemophiliacs. The VWF gene is associated with von Willebrand disease (comprising multiple types (1, 2A, 2B, 2M, 2N, and 3)) and can follow either an autosomal recessive, co-dominant or dominant inheritance pattern (James and Lillicrap, 2013, Br J Haematol.).

During normal coagulation, platelets form a plug at the site of injury in the blood vessel. Next, activation of a complex coagulation signaling cascade involving several clotting factors (such as factor VIII or factor IX, among others) culminates in the formation of fibrin strands that reinforce the platelet plug. When part of the cascade is impaired, as in hemophilia, then normal clotting is disrupted and bleeding occurs. This abnormal bleeding can be mild or severe, depending on the underlying defect, and is associated with increased morbidity and mortality. Damage sustained from brain or joint bleeds can be especially problematic. There is no long term cure. The current treatment for hemophilia involves replacing the defective coagulation factor using either blood-derived or recombinant factor VIII or factor IX. A major complication occurs if the patient develops antibodies (also called inhibitors) to the replacement factor.

Hemophilia is considered a rare disease with a prevalence of 1/5000-10,000 for hemophilia A, and 1/40,000 for hemophilia B. Both forms are more common in males than females since both genes reside on the X chromosome.

The severity of the bleeding symptoms in hemophilia A or hemophilia B as well as the likelihood of inhibitor development is related to the type of mutation. For example, while roughly half of severe hemophilia A cases are caused by a large inversion within intron 22, inhibitor development is more likely in patients carrying nonsense mutations or large deletions. Inhibitors develop in 25-30% of hemophilia A and 1-4% of hemophilia B patients.

VWD occurs due to qualitative or quantitative defects in vWF, a protein with an important role in platelet adhesion to wound sites. vWF also binds directly to factor VIII; unbound factor VIII is rapidly cleared from the body. Thus, type 2N VWD, the subtype defective in binding to factor VIII, can be misdiagnosed as hemophilia A, since both diseases feature low levels of factor VIII. Genetic testing can definitively distinguish between these two disorders. Genetic testing can also aid in the differential diagnosis of all the VWD subtypes: type 1, 2A, 2B, 2M, 2N, and 3. Treatment for VWD depends on severity of symptoms as well as subtype. Many mild cases do not need treatment.

The prevalence of VWD may be as frequent as 1/100; however, the great majority of those cases are mild or asymptomatic. The prevalence for clinically significant VWD is 1/10,000.

Current genetic methods for confirming a clinical diagnosis hemophilia or VWD takes at least a week and usually longer. Doctors and patients are always searching for faster testing turnaround times for faster patient diagnosis and management.

Therefore, there is a need for an improved method to rapidly identify mutations, polymorphisms and other variants of genes involved with hemophilia and VWD in order to identify, diagnose, treat, and assess the risk of an individual in developing one of these inherited bleeding disorders.

### SUMMARY

The present invention is directed to a method for determining a subject's risk for developing or being a genetic carrier for hemophilia A, hemophilia B, or VWD. The method includes the steps of obtaining a sample of genetic material from the subject, amplifing the genetic material using two or more primers specific for the genes underlying hemophilia A, hemophilia B or VWD, determining the DNA sequence of the amplified genetic material, and comparing the DNA sequence of the amplified genetic material with a DNA sequence from a normal control subject. One or more DNA sequence alterations in the amplified genetic material not present in the DNA sequence from the normal control subject indicates that the subject has a risk for developing hemophilia A, hemophilia B, or VWD. The DNA sequence alteration can be a mutation, a polymorphism, or a structural variant. In one aspect, at least three genes are amplified. In another aspect, the genes underlying hemophilia A, hemophilia B and VWD comprise F8, F9, and VWF. The subject's risk for developing or being a genetic carrier for hemophilia A, hemophilia B, or VWD can be determined within 48 hours of receipt of the sample of from the subject. In another aspect, the subject's risk for developing hemophilia A, hemophilia B, or VWD is determined within 5 days of receipt of the sample of from the subject.

The invention is also directed towards methods for diagnosing hemophilia A, hemophilia B, or VWD. The method includes the steps of obtaining a sample of genetic material from the subject, amplifing the genetic material using two or more primers specific for the genes underlying hemophilia A, hemophilia B or VWD, determining the DNA sequence of the amplified genetic material, and comparing the DNA sequence of the amplified genetic material with a DNA sequence from a normal control subject. One or more DNA sequence alterations in the amplified genetic material not present in the DNA sequence from the normal control subject indicates that the subject has a risk for developing hemophilia A, hemophilia B, or VWD. The DNA sequence alteration can be a mutation, a polymorphism, or a structural variant. In one aspect, at least three genes are amplified. In another aspect, the genes underlying hemophilia A, hemophilia B and VWD comprise F8, F9, and VWF. The diagnosis of hemophilia A, hemophilia B, or VWD can be determined within 48 hours of receipt of the sample of from the subject. In another aspect, the subject's risk for developing hemophilia A, hemophilia B, or VWD is determined within 5 days of receipt of the sample of from the subject.

### DESCRIPTION

According to one embodiment of the present invention, there is provided a method for rapid detection of mutations, structural variants and polymorphisms associated with hemophilia A, hemophilia B, or VWD. The time for diagnosing or determining the patient's risk of developing hemophilia A, hemophilia B, or VWD using the method of the present invention can be as early as 48 hours after receipt of the patient's sample. The method of the present invention can also be used to diagnose hemophilia A, hemophilia B, or VWD within 5 days after receipt of the patient's sample. The method involves analysis of three genes: Factor VIII (F8), Factor IX (F9), and Von Willebrand Factor (VWF), referred to herein as the “genes of interest.”

As used in this disclosure, except where the context requires otherwise, the term “comprise” and variations of the term, such as “comprising,” “comprises” and “comprised” are not intended to exclude other additives, components, integers or steps. Thus, throughout this specification, unless the context requires otherwise, the words “comprise,” “comprising” and the like, are to be construed in an inclusive sense as opposed to an exclusive sense, that is to say, in the sense of “including, but not limited to.”

As used in this disclosure, except where the context requires otherwise, the method steps are not intended to be limiting nor are they intended to indicate that each step is essential to the method or that each step must occur in the order disclosed.

As used herein, “sample” refers to any sample that can be from or derived a human patient, e.g., bodily fluids (blood, saliva, urine etc.), biopsy, tissue, and/or waste from the patient. Thus, tissue biopsies, stool, sputum, saliva, blood, lymph, tears, sweat, urine, vaginal secretions, or the like can be used in the method, as can essentially any tissue of interest that contains the appropriate nucleic acids. The sample may be in a form taken directly from the patient. Preferably, the sample may be at least partially purified to remove at least some non-nucleic acid material.

The term “DNA sequence” as used herein refers to chromosomal sequence as well as to cDNA sequence.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An “amplicon” is an amplified nucleic acid, e.g., a nucleic acid that is produced by amplifying a template nucleic acid by any available amplification method (e.g., PCR, LCR, transcription, or the like).

A “gene” is one or more sequence(s) of nucleotides in a genome that together encode one or more expressed molecules, e.g., an RNA. The gene can include coding sequences that are transcribed into RNA which may then be translated into a polypeptide sequence, and can include associated structural or regulatory sequences that aid in replication or expression of the gene.

A “set” or “pool” of primers or amplicons refers to a collection or group of primers or amplicons, or the data derived therefrom, used for a common purpose, e.g., identifying an individual with a specified genotype (e.g., risk of developing hemophilia). Frequently, data corresponding to the primers or amplicons, or derived from their use, is stored in an electronic medium.

A “structural variant” refers to a variation of a DNA sequence such as, for example, a deletion, duplication or inversion.

A set of oligonucleotide sequences, or primers, have been identified that facilitate the rapid identification of mutations, polymorphisms, and other variants associated with hemophilia and VWD. This set of oligonucleotides detects sequences that are indicative of whether an individual is a carrier for, or has hemophilia A, hemophilia B, or VWD.

The primers are used to amplify certain genes underlying hemophilia and VWD, including F8, F9, and VWF. This includes all exons of the three genes, plus 25 base pairs of flanking intronic DNA, the 5 prime and 3 prime untranslated regions of the genes, plus deep intronic and promoter regions associated with hemophilia A, hemophilia B, and VWD.

The DNA used in the method of the invention can be extracted from any source, such as, for example, whole blood, samples from a buccal swab, or it can be from previously extracted genomic DNA samples. Preferably, the DNA is extracted from whole blood using known techniques. Prior to analysis, extracted DNA can be stored for one month at room temperature or 2-8° C. For longer storage of up to 2 years, DNA can be stored frozen at <−20° C. to minimize the degradation of nucleic acid.

After extraction, the DNA sample is measured and assessed for purity. 30 ng of genomic DNA, at a minimum concentration of 1.7 ng/μL, is preferable for analysis, but as little as 1 ng is sufficient. The concentration of purified DNA should preferably be adjusted to between 2 ng/μL and 25 ng/μL prior to analysis. Optimal DNA purity is an absorbance ratio (A₂₆₀/A₂₈₀) of 1.80 or greater (typical range is 1.60 to 2.00).

The extracted sample DNA is then amplified. A well-known amplification method, for example, is the polymerase chain reaction (PCR). In PCR, a characteristic piece of the particular nucleotide sequence of interest is amplified with specific primers. If the primer pair finds its target site, a sequence of the genetic material undergoes a million-fold proliferation.

During the PCR process, the DNA generated is used as a template for replication. This sets in motion a chain reaction in which the DNA template is exponentially amplified. PCR can amplify a single or few copies of a piece of DNA by several orders of magnitude, generating millions or more copies of the DNA piece. PCR can be extensively modified to perform a wide array of genetic manipulations, as known by one of skill in the art.

In the method described herein, unique primer pool aliquots can be are used to amplify the sample of interest. The primer pool aliquots contain forward and reverse primer pairs SEQ ID NO. 1 through SEQ ID NO. 344, directed towards one or more of the three genes of interest, VWF, F8 and F9, as listed in Table 1.

TABLE 1

| SEQ | | SEQ | | |
|---|---|---|---|---|
| ID | Forward | ID | Reverse | |
| NO. | Primer | NO. | Primer | Gene |
| 1 | TCATGACTGC | 173 | GTATGCCATA | F8 |
| | TTTTGTACAA | | AAGCCTTTAT | |
| | ATCA | | GT | |
| 2 | AGGGCCATTG | 174 | CTATGCTCCC | F8 |
| | TTCAAATAT | | TTAGTCCT | |
| 3 | GAGCTTTCTG | 175 | CATTTCTTAC | F8 |
| | TTGCTACT | | AAGGAGCCAA | |
| | | | AA | |
| | | | A | |
| 4 | CCCTCTACTC | 176 | CACCAGAAGA | F8 |
| | CAGTCTCTA | | TACTACCTG | |
| 5 | TTCAGTTAGG | 177 | AAGCAAATAC | F8 |
| | ACCTTAAAAG | | TACTTTTTGT | |
| | AGTT | | CAG | |
| | GT | | TAA | |
| 6 | GCTGTGTGTT | 178 | AAAAACAAAG | F8 |
| | TCTTTGTTCT | | TGGTAGTAGG | |
| | ATTTG | | AA | |
| | | | AGG | |
| 7 | TGATCTGACT | 179 | CAAACAGACC | F8 |
| | GAAGAGTAGT | | TGGAAAAGTT | |
| | ACG | | | |
| 8 | CTATCTCACC | 180 | TCTTGAAACG | F8 |
| | AGAGTAAGAG | | CCATCAAC | |
| | ATTTC | | | |
| 9 | GTGGAGAGGA | 181 | GTAAAACGTT | F8 |
| | GGAGATGTAT | | GAGTACAGTT | |
| | | | CTT | |
| | | | GG | |
| 10 | TTCTCATTGT | 182 | CAGTACTTCA | F8 |
| | AGTCTATCTG | | AGATTTTAGG | |
| | TGTG | | TCA | |
| | | | TT | |
| 11 | CTCCAAGGTT | 183 | CCAAAAACAT | F8 |
| | AGAATGGCTA | | GAAACATTTG | |
| | AAG | | AC | |
| | | | C | |
| 12 | ATATGATTTA | 184 | TAATGAATAG | F8 |
| | GCCTCAAAGC | | CCAAGAAAGT | |
| | TGT | | TC | |
| | | | ATGG | |
| 13 | TCTTTGAGTC | 185 | TGATTCATGA | F8 |
| | CTACGTCCTT | | CAGAATGCTT | |
| | | | ATG | |
| | | | G | |
| 14 | TTACGTTTTG | 186 | CTGACATCAA | F8 |
| | TTTTCTTGGA | | AGCCAAGTT | |
| | ATTCA | | | |
| 15 | ACCCTCGATT | 187 | CTTCAGCTTG | F8 |
| | TGCATACG | | TTCGATACCA | |
| 16 | AAAGTGGGAA | 188 | CTAGGAAAAT | F8 |
| | TACATTATAG | | GAGGATGTGA | |
| | TCAG | | | |
| | C | | | |
| 17 | TGGAGAAAGG | 189 | GGAACAATTT | F8 |
| | ACCAACATA | | CATGAAAAGA | |
| | | | CC | |
| | | | AAAT | |
| 18 | TTGACACACT | 190 | ATGACAGTAG | F8 |
| | TTTTAGAACT | | CCCTAGAATA | |
| | AACA | | TC | |
| | GT | | AGTG | |
| 19 | GCATTCACAG | 191 | GGAATAAGAT | F8 |
| | CTGTTGGT | | AATGGGCATA | |
| | | | AC | |
| | | | ATAT | |
| 20 | GAAGGAACAC | 192 | GACATGTTTC | F8 |
| | AAATGCTAAC | | TTTGAGTGTA | |
| | T | | CAG | |
| | | | T | |
| 21 | CAAGTCTCAT | 193 | GATGAGAAAT | F8 |
| | TTGTCAAAGT | | CCACTCTGGT | |
| | GC | | TC | |
| | | | AT | |
| 22 | CCAGTGCCTA | 194 | GGAATCCTCA | F8 |
| | GACCATTT | | TAGATGTCAG | |
| | | | TTT | |
| | | | TT | |
| 23 | ACATACAATT | 195 | GCCATCGCTT | F8 |
| | CATTCATTAT | | TCATCATAGA | |
| | CTGG | | | |
| | AC | | | |
| 24 | CTGAAAATTT | 196 | AGTGCTGTGG | F8 |
| | GGTCATATAT | | TATGGTTAAG | |
| | CAAC | | A | |
| | CT | | | |
| 25 | TGGAATTAAG | 197 | CTGTAGCAAT | F8 |
| | TTTGTGGAAG | | GTAGATTCTT | |
| | CTAA | | CC | |
| | GA | | | |
| 26 | CTATATTCCT | 198 | TGTTTTCCAT | F8 |
| | GTACATTGTC | | TTCAGATTCT | |
| | CAGT | | CTA | |
| | | | CTT | |
| 27 | TGACAATTTT | 199 | CTGTTGAGAA | F8 |
| | GTGTCCTGAT | | ACGCACAA | |
| | AC | | | |
| 28 | TTTATTTCCT | 200 | CCAAATTTGG | F8 |
| | GACACAAGCA | | TAGGTCTACT | |
| | ACCA | | CTG | |
| | | | ATA | |
| 29 | CTACCACTCT | 201 | TCCAATTAAG | F8 |
| | CTGTTGACGA | | ATTAAATGAG | |
| | T | | AA | |
| | | | ACTG | |
| 30 | CACAAAGACC | 202 | AAGGACCTTA | F8 |
| | ATTTCTTTTA | | AGATCCTAGA | |
| | AACC | | AG | |
| | AA | | ATTA | |
| 31 | ATTGGAGACA | 203 | CTCCTTGCCT | F8 |
| | AGGCTGAA | | TGATTGAATC | |
| | | | ATA | |
| 32 | TAGCAGTTAG | 204 | TTCAAATTTG | F8 |
| | GGAAACATTC | | CCTCCTTGCT | |
| | TCT | | AA | |
| 33 | CAAGCAGGAT | 205 | TCAGAAGAAA | F8 |
| | TATTAGAAAA | | ATTGGACTGG | |
| | TACT | | T | |
| | CC | | | |
| 34 | GTGTAAGGAA | 206 | TCAGGTTAAG | F8 |
| | GCAGAAACAG | | CCTCATACGT | |
| | ACT | | TTA | |
| | TTA | | AAA | |
| 35 | CTCAGTCTCT | 207 | TTTTGGCATT | F8 |
| | CTCTCTCCAT | | CTTTTCCCAT | |
| | ATAAG | | TGA | |
| | | | CTA | |
| 36 | TCAAGTTTCT | 208 | GAGAAGACTG | F8 |
| | TCAACTCTGT | | ACCCTTGGTT | |
| | TGCT | | T | |
| 37 | TATGGCAGAC | 209 | GCAATTGTTG | F8 |
| | TGGAGTGTTT | | AAAGCTTTGA | |
| | | | AA | |
| | | | TAAA | |
| 38 | CTAGTTCCAT | 210 | AAAGGCAAAA | F8 |
| | GAACATTTGA | | GAATGGCTAC | |
| | GAAA | | TT | |
| | T | | | |
| 39 | GGAGTGTAAA | 211 | ATGGAAAACC | F8 |
| | TAAGTACAAA | | CAGGTTAGTT | |
| | TACT | | AT | |
| | GT | | TAT | |
| 40 | CATGCTTGAT | 212 | GCAGACACTG | F8 |
| | CAGGATAATA | | CCTTGAAG | |
| | AACA | | | |
| | AC | | | |
| 41 | CTTGGCCATC | 213 | ATCTGCAAAA | F8 |
| | ACAAATTTCA | | TGGAGAGAAT | |
| | AGAA | | AC | |
| | | | AAT | |
| 42 | AGAATGAAAC | 214 | TGGCTTGTTG | F8 |
| | CCAGCACTT | | TACTCTAATT | |
| | | | GAG | |
| | | | C | |
| 43 | AATAGTCAAA | 215 | TAGAACAGCC | F8 |
| | AAGTGCTTAC | | TAATATAGCA | |
| | CTGT | | AG | |
| | | | ACAC | |
| 44 | AGACATTTTG | 216 | AACGTTTTAG | F8 |
| | TATTTTAGGC | | AATCTGTGTT | |
| | ATAG | | ATG | |
| | GT | | AGT | |
| 45 | TGAGGTACTT | 217 | AAAGAGGAAA | F8 |
| | TTGAGTCACC | | CCAGGTGAGT | |
| | CAT | | T | |
| 46 | AGAAATGCAG | 218 | TGTCCTGTCA | F8 |
| | GACTGATGAT | | GACAACCA | |
| | AGTT | | | |
| 47 | TTGCATATAG | 219 | TTGCTACTTC | F8 |
| | TCCCATGACA | | AGTTCTTCCT | |
| | GT | | GT | |
| 48 | CAACAGTCTA | 220 | GGTGCAAAGA | F8 |
| | GAAATGCG | | GCGGGAT | |
| 49 | TGCCAGTGGA | 221 | AAGCTTTATG | F8 13i variants |
| | ACACGACAGA | | GCTTGATATA | |
| | | | ATC | |
| | | | CC | |
| 50 | TCCCATAATG | 222 | ATTTTTACCT | F8 1i del |
| | GAGCAGTAAT | | GCCACAGGTA | |
| | CAGC | | TAG | |
| 51 | GACCCTGAGG | 223 | GGAGAGAAAA | F8 5i two indels |
| | ATTGTTGA | | TCAGTTTGAG | |
| | | | C | |
| 52 | TGGCAAATGT | 224 | AATCAATCAT | F8 c.1010- |
| | ATTCAACATT | | TGTCCAGTTG | 842G > A |
| | CTCA | | GA | |
| | G | | | |
| 53 | GCACCCAGGT | 225 | ACTGCTCTCA | F8 c.-112G > A |
| | AGTATCTTC | | GAAGTGAATG | |
| 54 | ATAAAACTTA | 226 | ACATTATACA | F8 |
| | TCGCTATGTG | | CACATTTCTC | c.143 + 1400A > |
| | AATG | | ACTC | G |
| | c | | | |
| 55 | AGAGTCCAAA | 227 | TACATACTGG | F8 |
| | GAATAATGGG | | TGCTTTTACT | c.143 + 1567A > |
| | CA | | TCTGTT | G |
| 56 | CATGTGATTT | 228 | ATGCTGATTA | F8 c.144- |
| | ATTTGCCTGT | | ACAGGATAAG | 10758-10757 |
| | CTCA | | CTGA | insTATA |
| 57 | ACTACATGCC | 229 | CTTATTCATT | F8 c.144- |
| | ACTTTGCCAT | | CCACATCCTG | 1259C > T |
| | AAC | | GTA | |
| 58 | AATATCCTAG | 230 | TAATGTCACC | F8 |
| | ATCAGGAGCA | | CACTAAACTC | c.l443 + 329A > |
| | AGCA | | GACA | G |
| | A | | | |
| 59 | AATGTCATAA | 231 | GTTCCACAGT | F8 c.144- |
| | TTATCAGACC | | TTTTGGACAT | 3700C > T |
| | AGGA | | TC | |
| | A | | | |
| 60 | GAGAGAGAGA | 232 | GATAGGAAGA | F8 c.1444- |
| | ATGGAAGAGC | | ATGACTGGGT | 1833T > C |
| | AAT | | T | |
| | A | | | |
| 61 | GACACACCAA | 233 | CTACTACCCA | F8 c.144- |
| | GAGTCTCA | | TTCTAGATCC | 5714C > T |
| | | | CT | |
| 62 | CCAAAAGTGA | 234 | TATAGGAATC | F8 |
| | GAGTGAAACC | | CTCAGGTGAT | c.1537 + 325A > |
| | CT | | GTT | G |
| | | | TCA | |
| 63 | AGGTCATGAA | 235 | TACCATGTGA | F8 |
| | ATACAATATT | | CAATGAAGAT | c.1903 + 2536A > |
| | GCTG | | TC | G |
| | CT | | ACAA | |
| 64 | GAGTTGGTTG | 236 | GTCTACTTAA | F8c.2114- |
| | TTTGATGAAA | | TGGATGTCGA | 1681T > A |
| | AAGT | | ATT | |
| | CA | | cc | |
| 65 | AAATGCAATA | 237 | TCTGGGTTTG | F8c.2114- |
| | CCAGAAGAAG | | TGGTTGTTGT | 4382-4381 insG |
| | AGA | | T | |
| | AAG | | | |
| 66 | ACACTTTACA | 238 | CCAACTTTGC | F8c.2114- |
| | AAAATGTGCA | | TCTCTTTGAT | 6139C > T |
| | AGAC | | TTTT | |
| | CT | | AT | |
| 67 | AGCAACACAT | 239 | TGCATTTTCT | F8 |
| | AAAACACCGA | | GTCTTTTTGC | c.265 + |
| | | | TG | 333C > T |
| 68 | TTCCATAAAT | 240 | TGAGATAGAG | F8 |
| | ATTTGTGACT | | AGAAGAGAGG | c.5219 + 293T > |
| | GACT | | TT | A |
| | GA | | T | |
| 69 | ACTGTCACTT | 241 | TCTATTGTGC | F8 |
| | GTCTCCCAT | | TGACCACTT | c.5586 + 194C > |
| | | | | T; F8 c.5587- |
| | | | | 93C > T; F8 |
| | | | | c.5587-98G > A |
| 70 | GTACAGTGAA | 242 | CCATGGTAAT | F8c.5816- |
| | CACATGTCCA | | ATATTCCACA | 34A > T |
| | | | TCC | |
| | | | AA | |
| 71 | GTGATCAACA | 243 | GTGCTCTTTC | F8 |
| | TTATGAGTAG | | TTCTACCTAC | c.5998 + 182A > |
| | TGCT | | TCT | G |
| 72 | CTATTAAACT | 244 | TTTATTTTGG | F8 |
| | GGATGGTTTT | | TTCTTCACTG | c.5998 + 529C > |
| | T | | TCCC | T |
| | | | TT | |
| 73 | CAAACAGTGT | 245 | AAGCCCTGTA | F8 |
| | TAAGCTAGTT | | ACTTTTCTGC | c.5998 + |
| | T | | TC | 941G > A |
| 74 | ACCAAATTTG | 246 | TTTCTCAGCC | F8 c.5999- |
| | GTAGTTCCAC | | CTCAGTGTT | 277G > A |
| | T | | | |
| 75 | ACTCCGGCAG | 247 | CAAAATGCAA | F8 |
| | GTTTCTGTT | | AGCCCAGATA | c.601 + |
| | | | TTT | 1632G > A |
| 76 | ATGTTCCTCT | 248 | CTCTCTTCTG | F8 |
| | CTATTGATTC | | ATCCTTTCAG | c.6429 + |
| | T | | GT | 2788T > C |
| 77 | TTCAGCAAGA | 249 | CAAACCATAT | F8 c.6430- |
| | TAGATACAGA | | CAGTATCTAA | 4825T > C |
| | AGG | | AA | |
| | AAC | | GGAA | |
| 78 | GTTTTAACTT | 250 | CAGATAGCAA | F8 c.6575- |
| | TTGCACAGAT | | TAACAGAATT | 262_6575- |
| | TCTG | | GGCC | 192del |
| 79 | GATTGAAATG | 251 | ACTATAATCT | F8 |
| | CTAGAGTGAA | | ACTAGCCAAC | c.6900 + |
| | TTTG | | AC | 4104A > T |
| | T | | CAT | |
| 80 | GTCTATCTTT | 252 | AAATTGTTAT | F8 |
| | TCATGAGTTT | | GATACTGGAT | c.6900 + |
| | GTTGG | | TTG | 8517C > T |
| | A | | GCA | |
| 81 | AGTGCTGAGC | 253 | AGGGACCTTA | F8 c.6901- |
| | TACCTCTT | | ATGTTTCTCA | 2010A > G |
| | | | AA | |
| | | | AATT | |
| 82 | ACTTTGGAGC | 254 | AATGTCACCC | F8 |
| | AAAGGTCA | | AATAGCTCCA | c.6901- |
| | | | | 725T > C |
| 83 | TCATCTTTGC | 255 | CAATGGTTAT | F8 |
| | ATATCCCTTC | | GTAAACAGGT | c.787 + |
| | CA | | CT | 143T > C |
| | | | CT | |
| 84 | TCAGGTTTTC | 256 | TTAGAATTGC | F8 |
| | TTAACTCTTC | | CCATCAGGAG | c.787 + |
| | TGA | | CTT | 3221A > G |
| 85 | TTACCATGGC | 257 | ATAAATGCCA | F8 c.-905G > C |
| | CTAGGTCCTA | | GGTGGTTATG | |
| | | | AG | |
| | | | T | |
| 86 | TCATTTGAGA | 258 | GAACAAACTC | F9 |
| | ACTTTCTTTT | | TTCCAATTTA | |
| | TCA | | CCT | |
| | | | G | |
| 87 | CATTTCCAGA | 259 | TCTGCCTTTA | F9 |
| | AACATTCCAT | | GCCCAATT | |
| | TTCT | | | |
| 88 | ATACCCTTCA | 260 | CTGGCATAAC | F9 |
| | GATGCAGAGC | | CCTGTAGTAT | |
| | AT | | | |
| 89 | CAAAATTCTG | 261 | CATACTGCTT | F9 |
| | AATCGGCCAA | | CCAAAATTCA | |
| | A | | GTC | |
| | | | TAT | |
| 90 | ATCTTTAACA | 262 | AGCTGATCTC | F9 |
| | TTGCCAATTA | | CCTTTGTGGA | |
| | GGTC | | A | |
| | A | | | |
| 91 | ATGTATATTT | 263 | AAGGAAGCAG | F9 |
| | GACCCATACA | | ATTCAAGTAG | |
| | TGAG | | GA | |
| | T | | ATT | |
| 92 | TTAGAAACTC | 264 | CACCAATATT | F9 |
| | AGGAAGACAG | | GCATTTTCCA | |
| | GA | | GTT | |
| | | | TCA | |
| 93 | TTGTGAAGTT | 265 | TAACGACCAT | F9 |
| | AAATTCTCCA | | GGAGGGTAA | |
| | CTCT | | | |
| | GT | | | |
| 94 | TATGTCAACT | 266 | GTTGCCATAG | F9 |
| | GGATTAAGGA | | TGGAGAACCA | |
| | AAAA | | TA | |
| 95 | TAATACATGT | 267 | TGTCTAGTAA | F9 |
| | TCCATTTGCC | | AATAGCCTCA | |
| | AATG | | GTC | |
| | AG | | T | |
| 96 | CCCATTCTCT | 268 | GGCTGTTAGA | F9 |
| | TCACTTGT | | CTCTTCAATA | |
| | | | TTG | |
| 97 | TCTGGCTATG | 269 | TGAATTAACC | F9 |
| | TAAGTGGCT | | TTGGAAATCC | |
| | | | ATC | |
| | | | TTT | |
| 98 | AATCAGTTTT | 270 | ATAACCATAC | F9 |
| | TCTCTTTCTT | | AAGCTCTTAG | |
| | ACTCC | | AA | |
| | | | TGG | |
| 99 | CACACGCATA | 271 | TGCTTGGCTC | F9 |
| | CACACATATA | | TGAACACT | |
| | ATG | | | |
| 100 | AGACTTTGAG | 272 | ATACAGGGTG | F9 |
| | GAAGAATTCA | | ACTGATTCAC | |
| | ACAG | | AT | |
| | T | | C | |
| 101 | TAAATTGCTT | 273 | CTACAGACCT | F9 c. |
| | TGTGAGTGCC | | TTGGTCATT | *2545A > G |
| | TACT | | | |
| 102 | GTTACTTCAA | 274 | AATGATTCTT | F9 c. |
| | ATTTGAATGA | | ATATGGCAAC | *2864T > C |
| | CCAA | | TGC | |
| | AG | | AA | |
| 103 | AAAATAGTGC | 275 | AAATCACACA | F9 |
| | TGATAACAAG | | AATTAATTGC | c.520 + |
| | GTG | | TTT | 102_520 + |
| | GT | | GTG | 103del |
| 104 | TCCATCTTAT | 276 | TTTGCTGAAT | VWF |
| | TTGATCCTAA | | GCCACAAG | |
| | CTGGA | | | |
| | A | | | |
| 105 | CATACTGCAG | 277 | CAAGGCTTTC | VWF |
| | CACTGACA | | ATTTCAAAAG | |
| 106 | TTGCACTCCA | 278 | CTTCCCACCA | VWF |
| | TGGCATTG | | TTGTGAAG | |
| 107 | AATGTGGAGA | 279 | ACAACTATGC | VWF |
| | CCTCGAGATT | | CGCTGCTTT | |
| 108 | TGTGAATGGG | 280 | CTGCTAGCAC | VWF |
| | TTAGCATA | | CAGCTCTT | |
| 109 | GGCAGGCCTC | 281 | CACTGGGCTA | VWF |
| | GAAT | | TTTCCA | |
| 110 | CGCTCCTGAC | 282 | ATGGAAGATG | VWF |
| | ACATTTC | | TTCATCTAAG | |
| | | | GG | |
| | | | A | |
| ill | CCTCCAATTT | 283 | GGAGTATAGG | VWF |
| | CCCTCAAC | | CAGTGTGTGT | |
| 112 | AACTCAGTCT | 284 | AACACCACAG | VWF |
| | CTTCTTTCTT | | AACAAGTTCT | |
| | GG | | TT | |
| | | | GAG | |
| 113 | ATGCTTCCAG | 285 | TGGCTGGCTT | VWF |
| | TTTATTTCCC | | TATTTGGTTA | |
| | TTCT | | | |
| 114 | CTGGTCTCTG | 286 | CACGAGGATC | VWF |
| | GAATACAA | | AATCTTTTCT | |
| 115 | TTCTGGTGTC | 287 | GAATGGGTCT | VWF |
| | AGCACACT | | TGGCAATG | |
| 116 | GATTAGAACC | 288 | TACATGGTCA | VWF |
| | CGAGTCGTA | | CCGGAAATC | |
| 117 | AAGACTGAAC | 289 | GTCAAGCTGC | VWF |
| | ATAATGACTG | | TCACATTTA | |
| | AC | | | |
| 118 | GCAGGTCCTT | 290 | ATGGAGTTGT | VWF |
| | AAGGACAG | | AGGTTATGAG | |
| | | | AA | |
| | | | GG | |
| 119 | CCCACCTCCT | 291 | GGTGTCAACA | VWF |
| | TTCACACA | | GGAACATG | |
| 120 | TAGGCCATGA | 292 | AGTCATTGCT | VWF |
| | GGAGAAAG | | CTTCAGTGCT | |
| | | | A | |
| 121 | CTACTCACTT | 293 | GCTTGTAAAG | VWF |
| | CCTTGGAA | | ACTTTTTGGG | |
| 122 | CCAAGCCTTG | 294 | GTGCTGTGAT | VWF |
| | TAGCACTT | | GAGTATGAG | |
| 123 | ACCAACAGCT | 295 | AAATGCCCAG | VWF |
| | GGGTGAAA | | ACCAGTGA | |
| 124 | CAACCCAGAT | 296 | TCTCTGTCTC | VWF |
| | TCAGCTAG | | CATCATCATC | |
| | | | ACT | |
| | | | TAC | |
| 125 | AGAACCTTTC | 297 | TTATTTATTG | VWF |
| | TTACCCTTCC | | CCACATTCTC | |
| | TAAG | | AGC | |
| | A | | A | |
| 126 | TCTGCTTTAC | 298 | GTCACTTGGA | VWF |
| | AATGACTTGC | | GAACGTAC | |
| | CT | | | |
| 127 | AGTACTCCAG | 299 | CACCCAGAGT | VWF |
| | TAGAAACCAG | | ATTCTGTG | |
| | A | | | |
| 128 | AGACTCTAGC | 300 | CCACCAGCAG | VWF |
| | CACAGTTAGT | | ACCTAGAATT | |
| | TTTG | | | |
| | G | | | |
| 129 | AGCCCTTGTT | 301 | AGTCTCTTGA | VWF |
| | TCTTCCTCTC | | ATTTAGTCAC | |
| | T | | AGA | |
| | | | CT | |
| 130 | ATGTGCCTCA | 302 | TTTCATGGTT | VWF |
| | GACACTGA | | CTGCAGATTG | |
| | | | T | |
| 131 | TCTAGGTGCC | 303 | TTCAGCCTGC | VWF |
| | AGTGTTTG | | TTTTGTTTG | |
| 132 | AAGTTGCTAA | 304 | CCACCTTCCT | VWF |
| | AAAGGCAAAG | | GAGAGAAGAG | |
| | AAT | | | |
| 133 | CAAGACCTAG | 305 | TAATATTGGT | VWF |
| | AAGCACCTT | | GACGCCCATA | |
| | | | GT | |
| 134 | AGGCCAATCA | 306 | GATGATTAAC | VWF |
| | CTGGTGAA | | CATGTTGAAT | |
| | | | CA | |
| | | | GC | |
| 135 | CTCTCCTTGT | 307 | GAGAGGTTTG | VWF |
| | TCTCAGCA | | AGCTGATG | |
| 136 | GGAAGGCATG | 308 | TAGAAGGTGG | VWF |
| | TTAGTGAA | | GAGAGACA | |
| 137 | GCCGCACATA | 309 | AGGGAGACAC | VWF |
| | CGTGACA | | TAACGGA | |
| 138 | AGGTGGTTTT | 310 | TCTGCATGTA | VWF |
| | CCTGACAT | | GTAGGCAT | |
| 139 | TCTTTAATGG | 311 | GATCCTGTGA | VWF |
| | CTGTGCGTTA | | CACGTACT | |
| 140 | AATGGTCGGG | 312 | GTGGAGGCAG | VWF |
| | ATTGACAC | | CGAGTATA | |
| 141 | TCATGCACAG | 313 | GACGTCCATG | VWF |
| | AAAGCAAT | | CAGTTTTG | |
| 142 | TTTGGGTGGG | 314 | GACAGGGTAT | VWF |
| | TGATTTTT | | GAGAGTGAG | |
| 143 | TGAGCATATT | 315 | GGAGTTTCTG | VWF |
| | TAATATCAGC | | AATCATTCAG | |
| | CACA | | CT | |
| | ACA | | | |
| 144 | GGCAGAGAGT | 316 | GGCTCAAGTC | VWF |
| | AACCAGGTT | | TCAGACAA | |
| 145 | AAACGGAACG | 317 | TGCCCTTGTA | VWF |
| | AGAAAATGC | | CTCACGAAG | |
| 146 | ACGATCAGGG | 318 | AGAGTGGAGG | VWF |
| | AGCAGAAA | | GAGGATCT | |
| 147 | GCTCTGCTGT | 319 | CTTGCTGTCC | VWF |
| | TTTAGAGG | | AACATTCC | |
| 148 | AAGAAGCCAA | 320 | GAACTGACAA | VWF |
| | TACTGAACCA | | AAGCTGGTTG | |
| | AAC | | | |
| | T | | | |
| 149 | CAGTCAGTCC | 321 | CATTTCCTTT | VWF |
| | TGCATCTT | | CATTGTTTCC | |
| | | | TTTT | |
| | | | GG | |
| 150 | GCAGCCTCTT | 322 | TCGTCCTGGA | VWF |
| | GATCTC | | AGGAT | |
| 151 | AGCATCCAAG | 323 | ATGAATGTGC | VWF |
| | AGCCTCAGAG | | AGGAACTCT | |
| | T | | | |
| 152 | ACTGTGGAGT | 324 | CCTATAGCAT | VWF |
| | TGACACAG | | AGCTGAATAC | |
| | | | TTA | |
| | | | C | |
| 153 | ATGGCATAGA | 325 | CAGGGCCACT | VWF |
| | ATGTGGCT | | CAGTTTAT | |
| 154 | GCAGTATCTC | 326 | AATGGCTCTG | VWF |
| | ACACTGACA | | TTGTGTAC | |
| 155 | GAGGCGGATC | 327 | GAGGTCTTGA | VWF |
| | TGCTTG | | AATACACAC | |
| 156 | CTCTGTGTCC | 328 | GCTTGCTTCC | VWF |
| | ATACCACC | | TGGAATGTC | |
| 157 | ACTTTTTACC | 329 | CTTTTAGTTA | VWF |
| | CAAAACCTAG | | AAAATGAGGC | |
| | TCTCT | | TTC | |
| | A | | C | |
| 158 | GCCATCCAGT | 330 | GACTAAGAGC | VWF |
| | CCCTAC | | CAGAGTTC | |
| 159 | GTACATGAGA | 331 | CTATGTCTCC | VWF |
| | CAGGAAGC | | ACTGTTAACC | |
| 160 | CAGGTCTCTT | 332 | GCCTGTGTTC | VWF |
| | CCACTTTAA | | AATTCTAGGG | |
| 161 | TTCCAGGAAG | 333 | CCTCACCAGC | VWF |
| | CAAGCTCTA | | AGCAACCT | |
| 162 | GTTGAAGTCG | 334 | AGAAGAAGGT | VWF |
| | GCTTCAC | | CATTGTGATC | |
| | | | CC | |
| 163 | CTCTTGAATA | 335 | GCTGTATTCA | VWF |
| | CTATTTTTGT | | GATGCTGGAT | |
| | TTCTT | | ATA | |
| | TG | | A | |
| 164 | GAAGACCAGG | 336 | AGGTTCTTCC | VWF |
| | TCCAGTA | | TGAACCATT | |
| 165 | AGTCACTTAA | 337 | TTCCGTTTAG | VWF |
| | AAGCTGAATG | | GGCCT | |
| | ATTC | | | |
| | AGAA | | | |
| 166 | AGCCAAGCTG | 338 | TTCGTGCCCT | VWF |
| | AGCATTG | | AACAG | |
| 167 | GTCGATCTTG | 339 | TGCACGATTT | VWF |
| | CTGAA | | CTACTGC | |
| 168 | GGTGCAAATG | 340 | AGGTTCTCCG | VWF |
| | AACCGT | | AGGAGG | |
| 169 | ATCAGGTCTG | 341 | TCCTGGTTAT | VWF c.- |
| | CTACAGCT | | CCCACACA | 1522_- |
| | | | | 1510del |
| 170 | TCAGGGCAAG | 342 | GGAAATGTCC | VWF c.- |
| | CTGATACA | | TTGAGAATTA | 1873A > G; |
| | | | GA | VWF |
| | | | ATTA | c.-1886A > C |
| 171 | AACACCATCT | 343 | ATGGCCTTTT | VWF c.- |
| | GCTAAACTAA | | CTACTGTCT | 2485G > A |
| | TTCC | | | |
| 172 | CACCAGAGGC | 344 | GATGGCCTTT | VWF c.- |
| | AGAATCAG | | TCTTTTCTT | 2520C > |
| | | | | T; VWF |
| | | | | c.-2613A > G |

Following standard techniques, the amplified sequences, or amplicons, are purified and a library is made with the purified amplicons. The library contains multiple sequences from different areas of the three genes of interest. The library made from the amplicons is then amplified and sequenced.

The DNA sequence of the library may be determined by any suitable method. For example, the DNA sequence may be determined by Sanger sequencing (chain termination), pH sequencing, pyrosequencing, sequencing-by-hybridization, sequencing-by-ligation, etc. Exemplary sequencing systems include pyrosequencing (454 Life Sciences), Illumina (Solexa) sequencing, sequencing by ligation (SOLiD, Applied Biosystems), long read sequencing (PacBio or Oxford Nanopore Technologies) and Ion Torrent Systems' pH sequencing system.

After sequencing, the DNA sequence of the library is mapped with one or more reference sequences to identify sequence variants. For example, the base reads are mapped against a reference sequence, which in various embodiments is presumed to be a “normal” non-disease sequence. The Human genome (Hg19) sequence is generally used as the reference sequence. A number of computerized mapping applications are known, and include GSMAPPER, ELAND, MOSAIK, and MAQ. Various other alignment tools are known, and could also be implemented to map the DNA sequence of the amplicon library.

Based on the sequence alignments and mapping results, plus available information including, for example, information from human mutational databases and other reference databases, sequence variants in the amplified amplicons are identified. Copy number (ie, deletions or duplications) may be inferred by analyzing the relative amplicon read depths; deletions will have a statistically significant decrease in relative amplicon read depth spanning the deleted region while duplications will have a statistically significant increase in relative amplicon read depth spanning the duplicated region. Furthermore, any sequence variations, including mutations associated with hemophilia A, hemophilia B, or VWD, disease-associated polymorphisms, benign polymorphisms and other known variants of undetermined significance can be determined to be homozygous, heterozygous, or hemizygous. Any variations in the gene analyzed as compared to the normal control gene can be classified as pathogenic, predicted pathogenic, uncertain, predicted benign or benign, as recommended by the American College of Medical Genetics (ACMG).

The present invention also contains a set of primers, SEQ ID NO. 345 through SEQ ID NO. 359 that are used specifically to identify the F8 large intron 1 and intron 22 inversions. The primers used are listed in Table 2. These inversions are the cause of approximately half of the severe hemophilia A cases. Following standard techniques as described above, patient samples are amplified. The amplicons are then purified and run out on a 1% agarose gel. The banding pattern of the patient sample is compared to the banding patterns from known positive and negative controls. Distinctive gel banding patterns indicate whether the sequences are normal or have an intron 1 inversion and/or an intron 22 inversion. These patterns can further discern whether a detected inversion is present in the heterozygous or homozygous/hemizygous state. Any instrument capable of determining the size of the amplicons (which corresponds to the number of nucleotides) is sufficient for this inversion-detection assay.

TABLE 2

| SEQ | | |
|---|---|---|
| ID | | |
| NO. | Inversion primer sequence | Gene |
| 345 | ACGGTTTAGTCACAAGT | F8 |
| 346 | GTCACTTAGGCTCAG | F8 |
| 347 | TCAACTCCATCTCCAT | F8 |
| 348 | GTCTTTTGGAGAAGTC | F8 |
| 349 | CATTGTGTTCTTGTAGTC | F8 |
| 350 | ATTGCTTATTTATATC | F8 |
| 351 | CAACTGGTACTCATC | F8 |
| 352 | TTACAATCCAACACT | F8 |
| 353 | CCCCCAGTCACTTAGGCTCA | F8 |
| 354 | CTTTCAACTCCATCTCCAT | F8 |
| 355 | ACTGAACTTGTTTATCAAAT | F8 |
| | CTACGTGTC | |
| 356 | CATTGTGTTCTTGTAGTCAG | F8 |
| | AGTGTACT | |
| 357 | TCTTGAGTCTGCAACTGGTA | F8 |
| | CTCATC | |
| 358 | TGCTTCTCTTTCTGTGTACC | F8 |
| | CTTC | |
| 359 | GATTGCTTATTTATATCTCC | F8 |
| | AAG | |

### EXAMPLES

### Example 1

DNA was extracted from the whole blood of Patient 1 using known procedures. The extracted sample DNA was then PCR amplified. Primer pool aliquots, listed in SEQ ID No. 1 through 344, were used. Each primer pool aliquot contained between 57 and 58 sets of forward and reverse primers as listed in Table 1. In the present example, the three target genes, F8, F9 and VWF, were amplified and analyzed.

Following amplification of the DNA, the amplicons were purified. A library was then made with the purified amplicons. The library was amplified and sequenced. After sequencing, the sequence of the amplicons from the sample were compared to a normal, control reference sequence comprising the Human genome (Hg19) sequence.

A heterozygous mutation in the F8 gene was identified in Patient 1. The mutation was a nonsense mutation, c.2440C>T, p.Arg814Ter, generating a premature stop codon that prevents the rest of the protein from being translated. This rare mutation is absent in the normal population and is known to be associated with severe hemophilia A.

### Example 2

DNA was extracted from the whole blood of Patient 2 using known procedures. The extracted sample DNA was then PCR amplified. Primer pool aliquots, SEQ ID No. 1 through 344, listed in Table 1, were used. Each primer pool aliquot contained between 57 and 58 sets of forward and reverse primers. Each primer pool aliquot contained between 57 and 58 sets of forward and reverse primers. In the present example, the three target genes F8, F9 and VWF, were amplified and analyzed.

Following amplification, the amplicons were purified. A library was then made with the purified amplicons. The library was amplified and sequenced. After sequencing, the sequence of the amplicons from the sample were compared to a normal, control reference sequence comprising the Hg19 sequence.

A common polymorphism, c.580A>G, p.Thr194Ala, was detected in the F9 gene from Patient 2. This polymorphism is present in about 15% of the general population. The polymorphism was detected in 48% of the total reads, indicating this donor is heterozygous for this variant. This polymorphism does not correlate with hemophilia.

### Example 3

DNA was extracted from the whole blood of Patient 3 using known procedures. The extracted sample DNA was then PCR amplified. Primer pool aliquots, SEQ ID No. 1 through 344, listed in Table 1, were used. Each primer pool aliquot contained between 57 and 58 sets of forward and reverse primers.

Following amplification, the amplicons were purified. A library was then made with the purified amplicons. The library was amplified and sequenced. After sequencing, the sequence of the amplicons from the sample were compared to a normal, control reference sequence comprising the Hg19 sequence.

Patient 3 had a heterozygous, nonsense variant (c.100C>T, p.Arg34Ter) in exon 3 of VWF. This variant has been previously reported in patients with type 3 von Willebrand disease (Kakela, 2006, Mol Genet Metab; Kasatkar, 2014, PLoSOne; Xiong, 2015, Science; Liang, 2017, Thromb Haemost; Ahmed, 2019, Haemophilia). This variant has a minor allele frequency of 0.00001195, according to the gnomAD database, and is classified as pathogenic using the ACMG-AMP criteria (Richards, 2015, Nat Genet).

Patient 4 had a heterozygous, nonsense variant (c.880C>T, p.Arg294Stop) in exon 8 of F9. This variant, also known as Arg248Ter, is known to be associated with hemophilia B (Green, 1989, EMBO J; Wang, 1990, Thromb Haemost; Attali, 1999, Thromb Haemost; Li, 2014, Am J Hematol). The minor allele frequency of this variant has not been established. This variant has been classified as pathogenic in the ClinVar database (https://www.ncbi.nlm.nih.gov/clinvar/) and using the ACMG-AMP criteria (Richards, 2015, Nat Genet). The F9 gene is associated with hemophilia B, an X-linked recessive bleeding disorder.

Patient 5 had a heterozygous, missense variant (c.1537G>A, p.Gly513Ser) in exon 10 of F8. This variant has been observed in several hemophilia A patients (Johnsen, 2017, Blood Adv; Margaglione, 2008, Haematologica 93; Bogdanova, 2007, Human Mut; Liu, 2002, Thromb Haemost). The minor allele frequency of this variant has not been established. This variant was classified as likely pathogenic using the ACMG-AMP criteria. The F8 gene is associated with hemophilia A, an X-linked recessive bleeding disorder.

Patient 6 had a heterozygous, missense variant (c.1481T>G, p.11e494Ser) in exon 10 of F8. This variant does not have a minor allele frequency established. This variant has been observed in Hemophilia A patients (Johnsen, 2017, Blood Adv 1). A different mutation at the same locus (c.1481T>C, p.Ile494Thr) is known to be associated with Hemophilia A (Schwaab, 1995, Br J Haematol; Antonarakis, 1995, Hum Mutat), and is classified as pathogenic in the ClinVar database. This variant is classified as likely pathogenic using the ACMG-AMP criteria.

Patient 7 had a hemizygous, missense variant (c.2167G>A, p.A1a723Thr) in exon 14 of F8. This variant, also known as p.A1a704Thr, has been reported in many patients with mild to moderate hemophilia A (Higuchi, 1991, PNAS; Nair, 2016, Clin Appl Thromb Hemost; Guo, 2018, Clin Appl Thromb Hemost. This variant has a minor allele frequency of 0.0000055, according to the gnomAD database. This variant is classified as pathogenic using the ACMG-AMP criteria and the ClinVar database.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference in their entirety.

What is claimed is:

1. A method for determining a subject's risk for developing hemophilia A, hemophilia B, or von Willebrand disease (VWD), the method comprising the steps of:
(a) detecting a germline alteration in a group of genes associated with hemophilia A, hemophilia B or VWD, the group consisting of: human coagulation factor VIII (F8) gene, IX (F9) gene, and von Willebrand factor (VWF) gene, wherein a germline alteration is detected by analyzing the sequence of the F8, F9, or VWF genes in a sample derived from the subject, wherein the F8, F9, VWF genes are analyzed by amplifying the F8, F9, or VWF genes in the sample using a minimum of two sequences selected from the group consisting of SEQ ID NO. 1 through SEQ ID NO. 359;
(b) comparing the amplified F8, F9, or VWF genes from step (a) with amplified F8, F9, and VWF sequences from a normal control subject; and
(c) determining the subject's risk for developing hemophilia A, hemophilia B, or VWD, wherein one or more germline alterations of F8, F9, or VWF genes in the sample derived from the subject that are not present in the normal control indicates that the subject has a risk of developing hemophilia A, hemophilia B, or VWD.

2. The method of claim 1, wherein the DNA sequence alteration is a mutation.

3. The method of claim 1, wherein the DNA sequence alteration is a polymorphism.

4. The method of claim 1, wherein the DNA sequence alteration is a structural variant.

5. The method of claim 1, wherein the step of amplification of the sample derived from the subject comprises amplification of at least three genes.

6. The method of claim 1, wherein steps (a)-(c) are performed within 48 hours of receipt of the sample derived from the subject.

7. The method of claim 1, wherein steps (a)-(c) are performed within 5 days of receipt of the sample derived from the subject.

8. A method for diagnosing hemophilia A, hemophilia B, or von Willebrand disease (VWD) in a subject, the method comprising the steps of:
(a) detecting a germline alteration in a group of genes associated with hemophilia A, hemophilia B or VWD, the group consisting of: human coagulation factor VIII (F8) gene, IX (F9) gene, and von Willebrand factor (VWF) gene, wherein a germline alteration is detected by analyzing the sequence of the F8, F9, or VWF genes in a sample derived from the subject, wherein the F8, F9, or VWF genes are analyzed by amplifying the F8, F9, or VWF genes in the sample using a minimum of two sequences selected from the group consisting of SEQ ID NO. 1 through SEQ ID NO. 359;
(b) comparing the amplified F8, F9, or VWF genes from step (a) with amplified F8, F9, and VWF sequences from a normal control subject; and
(c) determining the subject's risk for developing hemophilia A, hemophilia B, or VWD, wherein one or more germline alterations of F8, F9, or VWF genes in the sample derived from the subject that are not present in the normal control indicates that the subject has hemophilia A, hemophilia B, or VWD.

9. The method of claim 8, wherein the DNA sequence alteration is a mutation.

10. The method of claim 8, wherein the DNA sequence alteration is a polymorphism.

11. The method of claim 8, wherein the DNA sequence alteration is a structural variant.

12. The method of claim 8, wherein the step of amplification of the sample derived from the subject comprises amplification of at least three genes.

13. The method of claim 8, wherein steps (a)-(c) are performed within 48 hours of receipt of the sample derived from the subject.

14. The method of claim 8, wherein steps (a)-(c) are performed within 5 days of receipt of the sample derived from the subject.

15. A method for determining a subject's risk for being a genetic carrier for hemophilia A, hemophilia B, or von Willebrand disease (VWD), the method comprising the steps of:
(a) detecting a germline alteration in a group of genes associated with hemophilia A, hemophilia B or VWD, the group consisting of: human coagulation factor VIII (F8) gene, IX (F9) gene, and von Willebrand factor (VWF) gene, wherein a germline alteration is detected by analyzing the sequence of the F8, F9, or VWF genes in a sample derived from the subject, wherein the F8, F9, or VWF genes are analyzed by amplifying the F8, F9, or VWF genes in the sample using a minimum of two sequences selected from the group consisting of SEQ ID NO. 1 through SEQ ID NO. 359;
(b) comparing the amplified F8, F9, or VWF genes from step (a) with amplified F8, F9, and VWF sequences from a normal control subject; and
(c) determining the subject's risk for developing hemophilia A, hemophilia B, or VWD, wherein one or more germline alterations of F8, F9, or VWF genes in the sample derived from the subject that are not present in the normal control indicates that the subject is a genetic carrier for hemophilia A, hemophilia B, or VWD.

16. The method of claim 15, wherein the DNA sequence alteration is a mutation.

17. The method of claim 15, wherein the DNA sequence alteration is a polymorphism.

18. The method of claim 15, wherein the DNA sequence alteration is a structural variant.

19. The method of claim 15, wherein steps (a)-(c) are performed within 48 hours of receipt of the sample derived from the subject.

20. The method of claim 15, wherein steps (a)-(c) are performed within 5 days of receipt of the sample derived from the subject.
